(19) United States
(12) Patent Application Publication
(10) Pub. No.: US 2014/0200549 A1
(43) Pub. Date: Jul. 17, 2014

(54) IV CATHETER FOR PREVENTING BACKFLOW

(71) Applicant: Matthew W. Norkunas, Denver, CO (US)

(72) Inventor: Matthew W. Norkunas, Denver, CO (US)

(73) Assignee: Matthew W. Norkunas, Denver, CO (US)

(21) Appl. No.: 14/235,130

(22) PCT Filed: Jan. 19, 2012
(86) PCT No.: PCT/US2012/021813

§ 371 Date: Jan. 27, 2014

Related U.S. Application Data
(60) Provisional application No. 61/512,029, filed on Jul. 27, 2011.

(51) Int. Cl. A61M 25/06; A61M 25/02; A61M 39/06

(52) U.S. Cl. CPC A61M 25/0606; A61M 39/06; A61M 25/02; A61M 25/0637
USPC 604/508; 604/167.03; 604/164.04

(57) ABSTRACT

The present invention provides an improved intravenous (IV) catheter that enables a hands free means of preventing backflow of blood. The disclosed device incorporates a flow back inhibitor that includes leaflets radially layered upon one another in a conical form. In open mode the leaflets allow fluid flow and in closed mode the leaflets inhibit fluid flow. The device is attachable to a patient by means of an attachment component.

### CROSS-REFERENCE TO RELATED APPLICATIONS

This application claims priority to the U.S. provisional application 61/512,029 filed on Jul. 27, 2011, the contents of which are incorporated in its entirety.

### FIELD OF THE INVENTION

The present invention relates to intravenous (IV) catheters. Specifically, the present invention relates to a product designed to allow a hands free means of preventing backflow of blood.

### BACKGROUND OF THE INVENTION

The IV catheters most commonly used today are thin, tapering plastic tubes that are introduced into a peripheral vein via a sharp, hollow needle. The hollow needle sits within the catheter, and is used to access the vein where the catheter will ultimately be placed and the needle removed. In essence, the needle is analogous to a sword in a tip-less sheath and the catheter is the sheath itself. When the needle hits the vein, blood flows through the needle and away from needle tip which is inside the vein. This is due to the low pressure system induced in the vein by a tourniquet or other blood flow restricting means. Blood flows down the length of the needle into a collection apparatus that is in communication with the needle. This collection apparatus is often called the “flash” reservoir, as the blood “flashes” into that reservoir, which is generally clear. When blood is seen in the reservoir, the operator is signaled to slide the catheter off of the needle and into the vein. The needle is then retracted out of the catheter, leaving just the hollow plastic catheter in the vein. At this stage, the catheter usually is attached to an intravenous-solution containing tubing via a Luer-lock system. After being attached to the IV-solution containing tubing, the catheter is affixed to the patient with a clear, adhesive dressing and tape. This is generally the last step in IV placement.

There are problems/issues associated with this generally used system and method. One such problem/issue is that the operator placing the IV must keep the IV from getting pulled out of the vein before affixing it to the patient. A second exemplary problem/issue is that the operator placing the IV must keep blood from flowing backwards through the catheter and into the operative field before the tubing can be hooked onto the catheter hub via the Luer Lock system. There are various techniques for both; however, these techniques have varying reproducibility from procedure to procedure. Not only does the operator face significant annoyance and potential failure of placement by having to keep one hand on the catheter at all times while occluding and affixing the catheter, but leakage of blood onto the operator or patient puts both patient and operator (and anyone else who comes in contact with that blood) at risk for blood borne illnesses.

What is needed is a system that allows the operator to place the IV into the vein and take both hands off of the patient without any backflow of blood into the field. This system should be standardized and reproducible.

### BRIEF DESCRIPTION OF THE DRAWINGS AND THE FIGURES

FIG. 1 is a side view of the device of the present invention prior to use/deployment.

FIG. 2A is an aerial/top view of the device of the present invention prior to use; FIG. 2B is an aerial/top view of the device of the present invention after deployment of the adhesive portion of the device (step 1); and FIG. 2C is an aerial/top view of the device of the present invention after deployment of the adhesive portion of the device (step 1) as well as after removal of the needle (step 2).

FIG. 3 illustrates a rear view of the flow back inhibitor of the present invention.

FIG. 4 illustrates the device of the present invention.

FIG. 5 illustrates a side view of the device of the present invention with wings deployed.

FIG. 6 illustrates a rear view of the device of the back flow inhibitor of the present invention, further illustrating deployed wings.

FIG. 7 illustrates a top view of the device of the present invention with needle removed from catheter.

FIG. 8 illustrates an aerial view of the device of the present invention with needle removed.

FIGS. 9 A and B illustrate a rear view of a further embodiment of the present invention before and after deployment.

FIG. 10 illustrates a side view of a further embodiment of the present invention before and after deployment and unrolling of bio-occlusive dressing.

FIG. 11 illustrates an unrolling means.

### DETAILED DESCRIPTION OF THE INVENTION

The present invention relates generally to medical methods, systems and kits. Particularly, the invention disclosed herein allows an operator to place an IV into a vein, firmly adhere the IV to the patient through the use of the disclosed adhesive component of the device, and take both hands off of the patient without any backflow of blood into the field. This system is standardized and reproducible. With both hands free and no blood flowing backwards out of the catheter, the operator can safely take the IV tubing and hook it up to the patient. The system uses many of the same components of the current IV catheter technology in conjunction with new components thereby providing a means to provide enhanced care to the patient.

In practice, the following steps occur;
The caregiver places the needle into the vein.
Once the needle is in the vein and the “flash” is seen in the reservoir, the catheter of the present invention is slid into place.
The caregiver begins to remove the needle from the catheter.
Before the needle is fully removed, an adhesive component of the device is placed or deployed such that it is in contact with the skin and adheres the device to the patient.
The needle is removed fully, the flow back inhibitor of the present invention blocks backflow.
The end of the IV tubing is attached to the catheter via a medical valve system.
The medical valve system is secured in place.

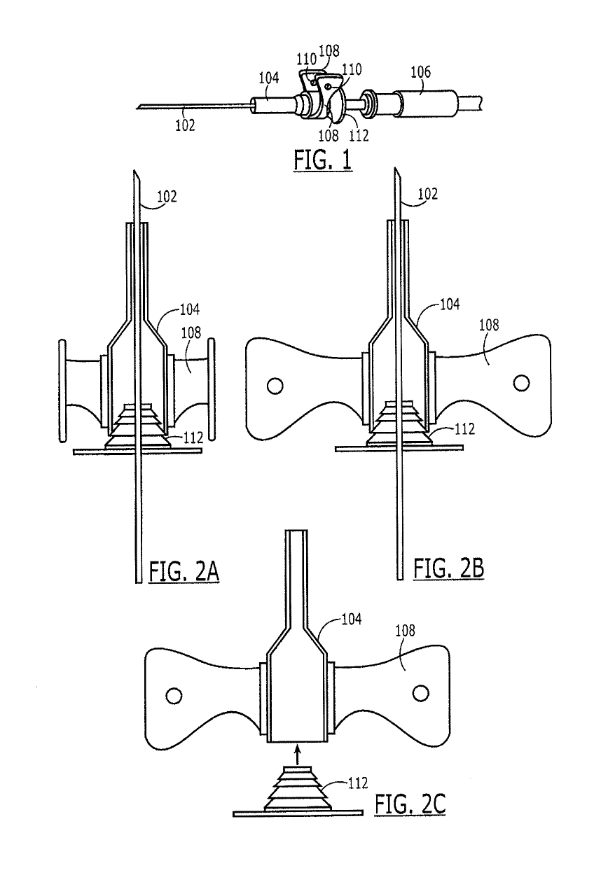

FIG. 1 illustrates an embodiment of the present invention. As shown, hollow needle 102 sits within the catheter 104. When the needle hits the vein, blood flows down the length of the needle into a collection apparatus or reservoir 104 in communication thereto. As illustrated herein, the catheter further comprises “wings”, wherein these wings serve as attachment component 108. In a further embodiment, each wing includes a hole or opening 110 allowing for suturing of the catheter to the skin. In another embodiment, the attachment component comprises skin hooks on the inferior surface with or without adhesive to allow for a more permanent fixation to the patient (not illustrated). In one embodiment the wings are semi-stiff with small hinges that connect the wings to the catheter. In operation, the hinges “snap” into place when they are either “up”/nondeployed or “down”/deployed. In an alternative embodiment, the adhesive component is flexible and can be rolled up against the catheter when not deployed, then unrolled and affixed after the catheter is in the vein. Once the needle is removed fully from the catheter the flow back inhibitor 112 blocks bloodflow backward through the needle. Thus, the catheter is secured and there is no bloodflow therethrough. The operator can now use both hands to attach the end of the IV tubing. The tip of the IV tubing goes through the flow back inhibitor 112 and the “leaflets” of the flow back inhibitor 112 that converge to keep blood from flowing out of the catheter are pushed up against the inner walls of the hub of the IV catheter, allowing for free-flow of the fluid through the catheter.

FIGS. 2A and B illustrate an embodiment of the present invention prior and after deployment of the adhesive component 108 of the device. FIG. 2C is an aerial/top view of the device of the present invention after deployment of the adhesive component of the device and after removal of the needle 102. As illustrated herein the flow back inhibitor 112 is oriented such that when the needle 102 is in place, the leaflets are pitched “forward” toward the needle tip (See FIGS. 2A and B). When the needle is withdrawn, the leaflets slide along the needle and quickly interlace or lock as the needle is retracted fully from the catheter. The leaflets maintain their slightly “forward” orientation, but with the needle out of the system, the leaflets are occlusive to bloodflow at low pressure. Essentially, the flow back inhibitor provides leaflets radially layered upon one another in a slightly conical shape. The “tip” of the cone is pointed into the catheter. (See FIG. 2C). The IV tubing can be inserted through the temporarily occluded flow back inhibitor area. The leaflets “fan” into the hub of the catheter, which pushes them into the walls of the inside of the catheter hub.

FIG. 3 illustrates the flow back inhibitor 112 in the occlusive to blood flow position.

A medical valve system such as a Luer-lock system allows for connection of the IV tubing to the catheter or to a component thereof. Such medical valve systems are known in the art for the transfer of fluid. The medical valve comprises a housing having an inlet, an outlet and a flow path defined therebetween. The medical valve also includes a first valve associated with the inlet wherein the first valve is adapted for receiving a male luer therethrough. The medical valve further includes a second valve normally substantially sealing the flow path distal to the first valve. The second valve is movable to open flow through the flow path by the action of the insertion of a male luer into the inlet. In one exemplary embodiment of the present invention the second component or flow back inhibitor of the catheter receives this male luer and thereby allows for flow therebetween. The medical valve system is securable to the catheter. Multiple means of securing the medical valve system to the catheter are know by those of ordinary skill in the art.

FIG. 4 illustrates the device of the present invention.

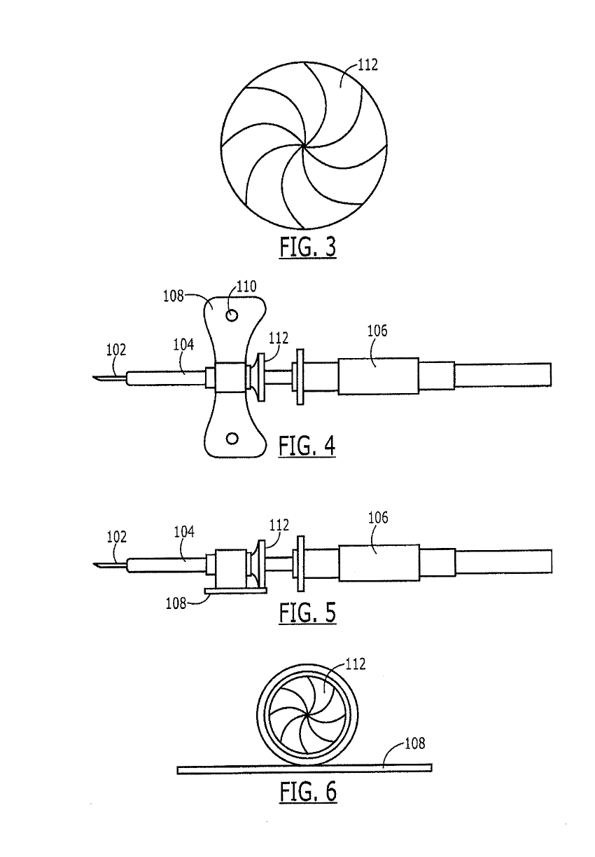

FIG. 5 illustrates a side view of the device of the present invention with wings deployed.

FIG. 6 illustrates a rear view of the device of the back flow inhibitor of the present invention, further illustrating deployed wings.

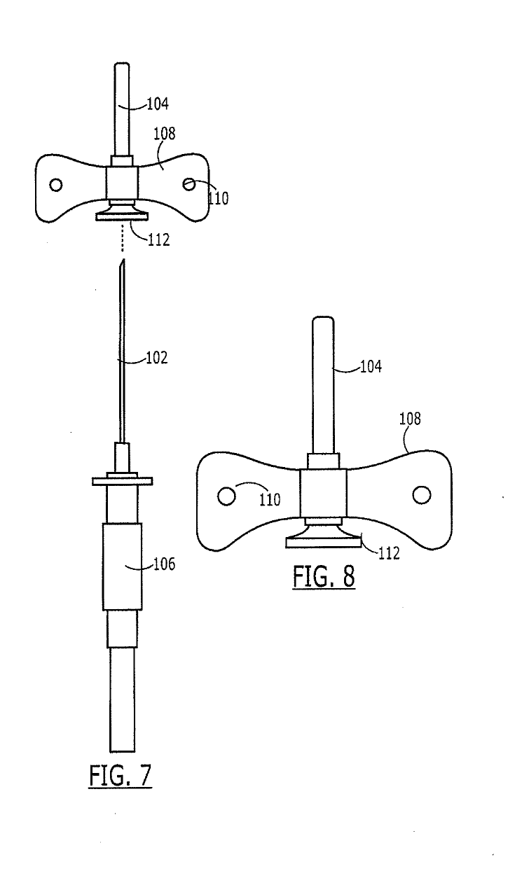

FIG. 7 illustrates a top view of the device of the present invention with needle removed from catheter.

FIG. 8 illustrates an aerial view of the device of the present invention with needle removed.

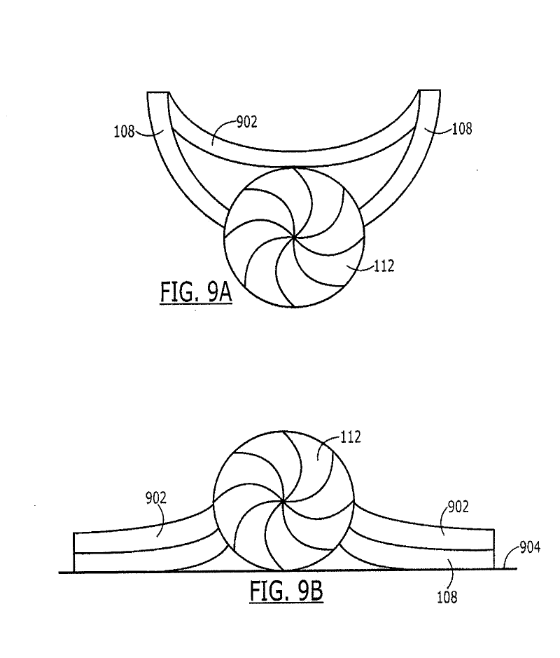

FIGS. 9 A and B illustrate a rear view of a further embodiment before and after deployment. Between the two adhesive components 108 is a bio-occlusive dressing 902. Such bio-occlusive dressing is a one-sided, sticky, sterile film. This film can be rolled up such as a “Ioban” dressing or unrolled such as “Tegaderm”. In this embodiment of the present invention the bio-occlusive dressing 902 is rolled up and fastened to the insides of the tips of the adhesive component 108, where the width of the rolled up dressing is the same width as the wings of adhesive component 108. Prior to deployment of adhesive component 108, the bio-occlusive dressing 902 is folded in half between the non-deployed “wings” of said adhesive component 108 Once adhesive component 108 is deployed and the adherent “wings” contact skin 904 and stick to the patient, bio-occlusive dressing 902 can be “unrolled” in a forward direction.

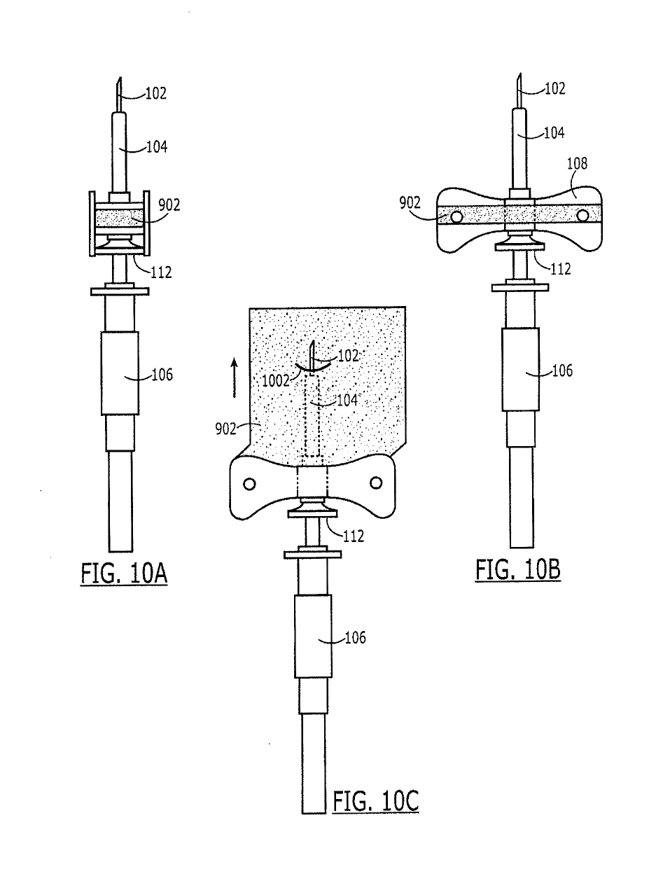

FIG. 10 illustrates skin entry site 1002 and unrolling bio-occlusive dressing 902 in a forward direction 1004.

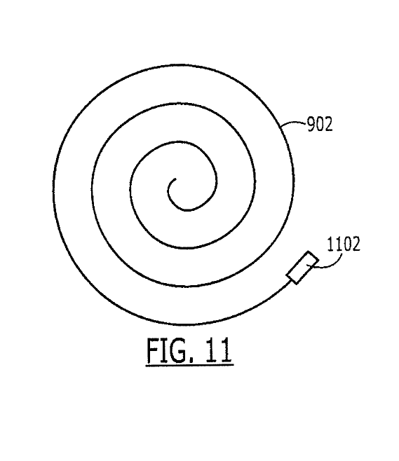

FIG. 11 illustrate an attached tag 1102, such as a paper tag to a nonsticky portion of bio-occlusive dressing 902. (The nonsticky portion is thrown away.) Pulling on the tag simply unrolls bio-occlusive dressing 902 like a carpet, leaving the sticky side of the bio-occlusive dressing 902 exposed downward toward the skin where it can easily be affixed.

The occlusive dressing is preferably long enough to easily cover beyond the point where the IV breaks the skin and goes into the vein.

Having now described a few embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention and any equivalent thereto. It can be appreciated that variations to the present invention would be readily apparent to those skilled in the art, and the present invention is intended to include those alternatives. Further, since numerous modifications will readily occur to those skilled in the art, it is not desired to limit the invention to the exact construction and operation illustrated and described, and accordingly, all suitable modifications and equivalents may be resorted to, falling within the scope of the invention.

What is claimed is:

1. An intravenous catheter comprising:
a removable hollow needle;
a catheter tube, wherein said catheter tube surrounds at least a proximal portion of said hollow needle;
a collection reservoir, wherein said collection reservation is in communication with said hollow needle;
an attachment component, wherein said attachment component provide a means of attachment of said catheter tube to skin; and
a flow back inhibitor comprising leaflets, wherein in open mode said leaflets allow fluid flow and in closed mode said leaflets inhibit fluid flow.

2. The intravenous catheter of claim 1, said attachment component comprising at least two wings.

3. The intravenous catheter of claim 2, said wings each comprising an opening, wherein said opening allows for suturing to said skin,

4. The intravenous catheter of claim 2, said wings each comprising skin hooks on an inferior surface.

5. The intravenous catheter of claim 2, said wings each further comprising an adhesive.

6. The intravenous catheter of claim 2, said wings further comprising a hinge, wherein said hinge connects said wings to said catheter, allowing movement in one plane; and wherein said wings are stiff.

7. The intravenous catheter of claim 2, wherein said wings are flexible and rollable.

8. The intravenous catheter of claim 1, wherein said flow back inhibitor is in open mode when said hollow needle is in place, and in closed mode when said hollow needle is removed.

9. The intravenous catheter of claim 1, wherein said hollow needle is removed, further comprising:
IV tubing, wherein said IV tubing is inserted through said flow back inhibitor, and wherein said flow back inhibitor is in open mode.

10. The intravenous catheter of claim 1, said flow back inhibitor comprising leaflets radially layered upon one another in a conical form.

11. The intravenous catheter of claim 10, said flow back inhibitor comprising a tip, wherein said tip is oriented toward said catheter.

12. The intravenous catheter of claim 9, wherein the IV tubing is connectable to said catheter.

13. The intravenous catheter of claim 12, wherein the IV tubing is connectable via a luer-lock system.

14. The intravenous catheter of claim 1, further comprising a bio-occlusive dressing, wherein said bio-occlusive dressing is rollable, and once rolled, is fastenable to said attachment component.

15. A method of placing an IV catheter comprising:
placing the needle of the intravenous catheter of claim 1 into a vein of a patient in need thereof;
sliding the catheter tube into a desired location once the “flash” is seen in the reservoir;
begin removing the needle from the catheter'
placing the attachment component such that it is in contact with the skin and adheres the catheter to the patient; and
completing removal of the needle.

16. The method of placing an IV catheter of claim 15, said IV catheter further comprising IV tubing, said method further comprising:
attaching an end of the IV tubing to the catheter via a medical valve system.

17. The method of placing an IV catheter of claim 16, further comprising:
securing the medical valve system.

18. The method of placing an IV catheter of claim 16, wherein said end of said IV tubing is inserted through the flow back inhibitor, wherein said insertion causes said leaves of said flow back inhibitor to be in open mode.

19. The method of placing an IV catheter of claim 18, said IV catheter further comprising a bio-occlusive dressing, wherein said bio-occlusive dressing is rollable, and once rolled, is fastenable to said attachment component, said method further comprising:
unrolling said bio-occlusive dressing; and
affixing said bio-occlusive dressing to the skin.

20. An intravenous catheter comprising:
a removable hollow needle;
a catheter tube, wherein said catheter tube surrounds at least a proximal portion of said hollow needle;
a collection reservoir, wherein said collection reservation is in communication with said hollow needle;
an attachment component, wherein said attachment component provide a means of attachment of said catheter tube to skin, said attachment component comprising at least two wings;
a flow back inhibitor comprising leaflets radially layered upon one another in a conical form, wherein in open mode said leaflets allow fluid flow and in closed mode said leaflets inhibit fluid flow, and wherein said flow back inhibitor is in open mode when said hollow needle is in place, and in closed mode when said hollow needle is removed; and
bio-occlusive dressing, wherein said bio-occlusive dressing is fastenable to said attachment component.
